(19) United States
(12) Patent Application Publication
(10) Pub. No.: US 2013/0327328 A1
(43) Pub. Date: Dec. 12, 2013

(54) APPARATUS AND METHODS FOR DIRECT INHALATION OF SCENTS AND MEDICINES

(71) Applicant: Greg Fisher, Boston, MA (US)

(72) Inventor: Greg Fisher, Boston, MA (US)

(21) Appl. No.: 13/686,268

(22) Filed: Nov. 27, 2012

Related U.S. Application Data
(63) Continuation of application No. 12/841,487, filed on Jul. 22, 2010, now abandoned.
(60) Provisional application No. 61/227,890, filed on Jul. 23, 2009.

(51) Int. Cl. A61M 15/08

(52) U.S. Cl. CPC A61M 15/08
USPC 128/203.12

(57) ABSTRACT

A dissolvable intranasal tab may provide temporary, controlled and direct inhalation of scents and/or medicine into the nose, thereby increasing the impact and velocity scents and/or medicine may have on the mind and body. The tabs may provide increased mobility, convenience, speed, and effectiveness of aromatherapy and medicinal inhalation therapy. The tabs may be substantially flat, triangular, and designed to be inserted into the nose allowing air to flow over both sides of the tab. The tab may be comprised of material relative to alternative components of the tab such that scents and/or medicines are formed into the shape of the tab and the outside of the tab is relatively dry to the touch. The tabs are adapted to be inserted separately into either nostril and to dissolve over a period of time inside the nose during normal breathing.

### CROSS-REFERENCE TO RELATED APPLICATIONS

This application is a continuation-in-part of U.S. patent application Ser. No. 12/841,487, filed Jul. 22, 2010, currently pending, which claims the benefit of priority of U.S. Provisional patent application No. 61/227,890, filed Jul. 23, 2009, the contents of which are herein incorporated by reference.

### BACKGROUND OF THE INVENTION

The present invention relates to intranasal delivery apparatus and methods and, more particularly, to apparatus and methods for the direct inhalation of scents and/or medicines into a person's airway.

The human nose is an under-optimized interface between the human body and external environment. The human nose can distinguish between hundreds of substances, even in minute quantities, while the human tongue, on the other hand, can only distinguish among five qualities of taste. The sense of smell is the only one of the traditional five senses without a commonly utilized and mobile device that closely and directly interacts with that sense. The other four senses have widely available devices or mechanisms to enhance, complement or manipulate each of those senses—glasses for sight, oven mitt for touch, spices for taste, and hearing aids for hearing are just some examples.

Aromatherapy is a form of alternative treatment to influence mood, cognitive function and/or health through one's sense of smell. While specific efficacy of aromatherapy remains unproven, some preliminary clinical studies show positive results and other studies are currently underway. Currently available aromatherapy methods lack undetectable intranasal apparatus that administer mobile delivery at close proximity to one's airway.

Pharmaceutical companies are testing ways to deliver vaporized or atomized drugs through the nose. Currently available medicinal nasal delivery methods may provide a burst of drug substance without a way to slowly release the drug intranasally.

Existing intranasal delivery devices may be two-piece devices held together by a connector. The connector must always be somewhat visible since it connects externally below the septum. Visibility while in use may be an obstacle to broad acceptance in the marketplace. Existing devices may not optimize air flow into and out of the nose. Instead, they do not dissolve and may fill a significant portion of the nasal space and restrict air flow, which is counterproductive, unless the device is intended to be used as a nasal filter. Existing devices may also lack the functional ability to allow sufficient amounts of scents or medicine to be diffused over a long period of time.

As can be seen, there is a need for an effective, dissolvable and undetectable apparatus and method to deliver optimal amount of scents and/or drugs directly into one's nasal passage over a period of time.

### SUMMARY OF THE INVENTION

In one aspect of the present invention, a nasal delivery device comprises a triangularly shaped tab designed from certain materials to allow scents or medicines infused into the dissolvable tab to evaporate into one's airway.

In another aspect of the present invention, the internal components of the tab are made from material to maximize the amount of scents or medicine absorbed into the tab while the external of the tab is designed to remain relatively dry to the touch.

In another aspect of the present invention, the triangular tab is substantially flat to allow air to flow over both sides of the tab when inserted into the nose.

These and other features, aspects and advantages of the present invention will become better understood with reference to the following drawings, description and claims.

### BRIEF DESCRIPTION OF THE DRAWINGS

FIG. 1 is a top view of an intranasal tab according to an embodiment of the present invention;

FIG. 2 is a side view of the intranasal tab of FIG. 1;

FIG. 3 is a kit designed to contain a number of the intranasal tabs of FIG. 1;

FIG. 4 shows a side-view of the tab of FIG. 1 inserted into a user's nostril; and

FIG. 5 shows a view of the inserted tab in a user's nostril from below.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides for direct inhalation of aromas and/or medicines into the nose, thereby affecting the impact scents and/or medicines may have on the mind and body. Embodiments of the present invention may provide increased mobility, convenience, speed, and effectiveness of aromatherapy and medicinal therapy.

The apparatus of the present invention may be a substantially flat, triangular tab that may be made from a combination of materials. The center of the tab may contain highly concentrated material with optimally absorbed scents and/or medicines thereinto, and with the external surfaces of the tab relatively dry as compared to the central portion of the tab. This may optimize passive diffusion of the infused scents or medicines from the higher density core to the lower density exterior while the water soluble tab dissolves.

The tab of the present invention may be less complex and better designed than conventional intranasal delivery devices. Embodiments of the present invention may include single, contiguous tabs that may be inserted separately into one or both nostrils. Since there is no connector to join two tabs, as is included in conventional intranasal devices, the tabs of the present invention may be entirely invisible once inserted. The substantially flat design with pliable perimeter edges may allow for better air flow into and out of the nose. The external surfaces of the tab are relatively dry to touch which reduces scents and/or medicines from coming in direct contact with the skin and the potential for irritation of the nose while the scents and/or medicine evaporate inside the nose. The triangular tab may also include rounded corners, allowing the tab to fit comfortably inside the nose and preventing the tab from being inserted too far or falling out of the nose while in use.

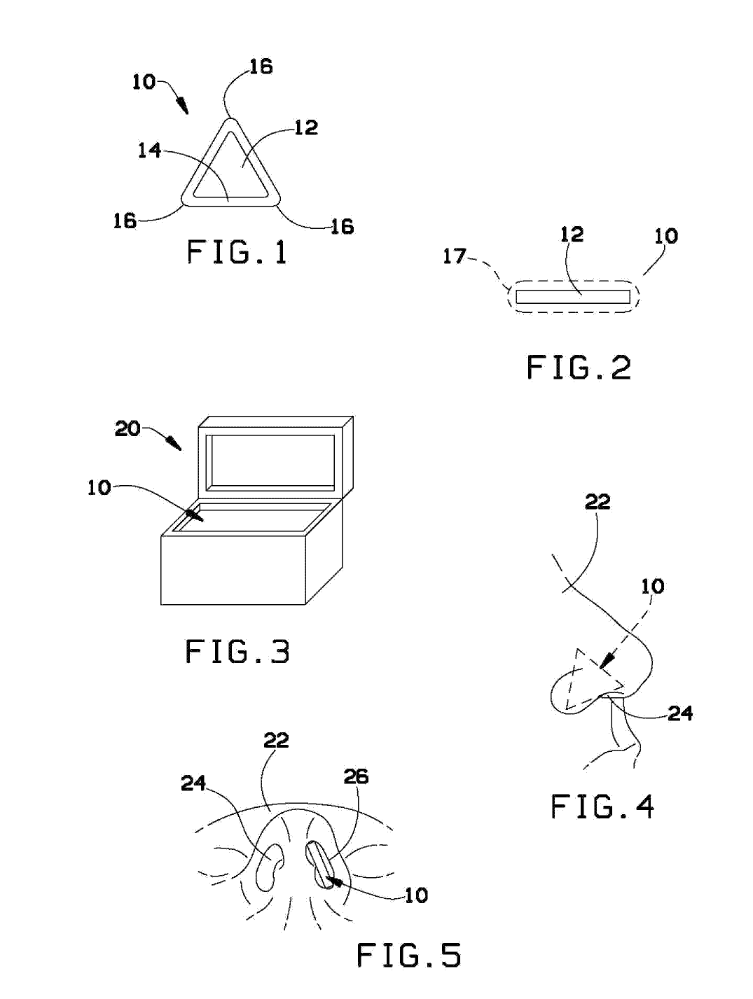

Referring to FIGS. 1, 2, and 4 an intranasal tab 10 has an equilateral triangular shape so that any of the three corners may be inserted up into the nose. Each side of the tab 10 may be from about 0.5 to about 1.0 inch, typically about 0.60 inch. The tab 10 may be from about 0.005 to about 0.015 inches thick, typically about 0.007 inches thick. The tab 10 may be formed from a combination of scents and/or medicines and water soluble materials that dissolve over a period of time.

The tab 10 may include a concentrated core 12 surrounded by a more pliable or thinner perimeter edge 14. The core 12 and perimeter 14 may be formed from multiple types or concentrations of the same or different material, mainly natural or synthetic water soluble polymers, and with the perimeter 14 being made thinner and/or from different concentrations of materials that are pliable. The perimeter edge 14 is better able to be molded to fit the contours of one's nasal cavity than the core 12. In preferred embodiments, the tab 10 can be made of a combination of material that can dissolve in about 30 minutes of intranasal use, specifically designed to dissolve inside the nose given the unique environment inside the nasal cavity with respect to humidity, temperature, mucus and air pressure. Of course, dissolve time can vary with temperature, humidity, mucus, air pressure and the like.

The same material or concentration of material used in the perimeter 14 may also be present in the exterior surface 17 of the tab 10. When inserted into the nose, the relatively dry exterior surface 17 may avoid irritation to the nasal cavity by minimizing the scented oil/fragrance or medicine from coming into direct contact with the mucus membranes of the inner nose until the tab interacts with the moisture and air pressure inside the nose and the tab dissolves and evaporates naturally into the airstream. The released vapors penetrate deeper into the sinus cavity for absorption inside the nose and for olfactory stimulation. Air is able to pass through the core 12 as well as the perimeter edge 14 of the tab given that there are small perforations made throughout the tab to aid breathability and increase dissolve time.

As discussed above, the tab 10 may be flat having an equilateral triangular shape which allows for any of the three corners of the tab to be inserted up into the nose. The tab 10 may have rounded corners 16 which may allow a natural and comfortable fit inside the nose. The outside perimeter edge 14 of the tab 10 may be pliable so the tab 10 can easily mold into the nostril. Referring to FIG. 5, the tabs 10 may be designed to fit snugly into the lower part of the nose so that when a tab is inserted into the nose, the opposite edge of the corner inserted into the nose may be flush with the opening of the nose. Once inserted, the tab 10 may not be visible in the nose (unless looking directly up into the nasal cavity). The slim design of the tab allows the tab to be placed in the center of the nostril so air can pass over both sides of the tab. This allows scents/medicines to be dispersed from both sides of the tab while also improving breathability.

Essential oils, fragrances or medicines may be infused into the tab. Natural and/or artificial scents may be used. The scents, fragrances and/or medicines may be gradually released over a period of 30 minutes. The specific dose amounts of each scent and/or medicine may depend on the individual scent and/or medicine, as well as how the tabs are marketed—for example, as regular strength or extra strength. The tabs can provide a longer residency time in the nose as compared to one-time burst medicines that may be sprayed or inhaled into the nose. The delivery method of the present invention may also provide increased bioavailability and efficacy. Moreover, the tabs can create less waste than nose drops and nasal sprays.

The tabs of the present invention, as discussed above, can be made from various materials. As a non-limiting example, the tabs can be made from various materials, the majority of which is typically a dissolvable water soluble polymer, which may be natural or synthetic, including but not limited to pullulan, hydroxypropyl cellulose, hydroxypropyl methylcellulose, or polyvinyl alcohol. Other composition and formulation materials can include essential oils and/or active pharmaceutical ingredients, glycerin fatty acid ester, reduced malt sugar syrup, glycerin, carrageenan, excipients and optimizers, which can be various combinations of super-disintegrants, surfactants, plasticizers, fillers, enzymes and other substances that balance the right rigidity, aroma and/or medicinal impact and speed of dissolvability inside the nose.

In some embodiments, CO₂ bubbles can be included. Enzymes, such as amylase or pullulanase may be added to help the polysaccharide polymer break down faster. Effervescent agents, such as sodium bicarbonate or sodium lauryl sulfate and citric acid can be used, as well as additional plasticizers and fillers, such as poloxamer and alkylpolyglycosides.

Examples of essential oils that can be used include citrus oils, such as lemon, orange, lime, grapefruit, and the like, highly sensorial oils such as vanilla, coffee, cocoa, green tea, tobacco, hops, and the like, and botanical oils, such as eucalyptus, lavender, rose, peppermint, pine and the like, as well as unique combinations of multiple oils.

Referring to FIG. 3, a kit 20 may include individually wrapped tabs 10 in a container. The kit 20 may include, for example twelve tabs 10, individually wrapped in cellophane or foil, for example, to maintain freshness and reduce the potential for premature evaporation. The kit 20 may contain tabs of a single fragrance or multiple fragrances, for example. A purchaser may be able to select a certain scent and/or medicinal vapor therapy based on the mood sought to be achieved at any particular time. Tabs may be selected for mood, for energy, better breathing, to complement food and beverages, or the like.

Referring to FIG. 4, a user 22 may insert the tab 10 into a first nostril 24. In addition, referring to FIG. 5, the user 22 may optionally insert a second tab 10 into a second nostril 26. Once inserted, the tabs 10 may not be visible from an ordinary view of the user 22. When the user 22 tips their head back, one may view the tabs 10 inserted into each nostril 24, 26.

It should be understood, of course, that the foregoing relates to exemplary embodiments of the invention and that modifications may be made without departing from the spirit and scope of the invention as set forth in the following claims.

What is claimed is:

1. A nasal delivery device comprising:
a triangular tab formed of materials adapted to dose and disperse one or more scents and/or medicines directly inside the nose with the device inserted intranasally; wherein
the tab is formed of materials adapted to dissolve over a period of time inside the nasal cavity during use.

2. The nasal delivery device of claim 1, wherein the triangular tab is flat with pliable perimeter edges that conform to the nasal cavity allowing the tab to be positioned in the middle of the nostril so air flows over both sides of the tab.

3. The nasal delivery device of claim 2, wherein the length of each side of the tab is between 0.5 and 1.0 inch long and the thickness of each tab is from about 0.005 to 0.015 inches thick.

4. The nasal delivery device of claim 3, wherein the length of each side of the tab is about 0.60 inches.

5. The nasal delivery device of claim 3, wherein the thickness of the tab is about 0.007 inches.

6. The nasal delivery device of claim 3, wherein the length of each side of the tab is the same, wherein any of the three corners of the tab is equally designed to be inserted first into the nose.

7. The nasal delivery device of claim 3, wherein the materials are formed from certain scents and/or medicines, one or more water soluble dissolvable polymers and various excipients, surfactants, plasticizers, fillers and enzymes adapted to disperse scents and/or medicines inside the nose by dissolving intranasally over a period of time.

8. The nasal delivery device of claim 3, wherein the tab includes an exterior having a lower concentration of at least one of the scents and/or medicines.

9. The nasal delivery device of claim 3, wherein the perimeter edges of the tab are thinner or formed of less concentrated material than the core of the tab.

10. The nasal delivery device of claim 3, wherein the corners of the tab are rounded.

11. The nasal delivery device of claim 3, wherein the tab is perforated to allow air to flow through the tab.

12. The nasal delivery device of claim 3, wherein the tab is made from one or more of water soluble dissolvable polymers pullulan, hydroxypropyl cellulose, hydroxypropyl methylcellulose, polyvinyl alcohol, scents, medicines, excipients, surfactants, plasticizers, fillers, glycerin, glycerin fatty acid esters, reduced malt sugar syrup, and carrageenan.

13. The device of claim 12, wherein the tab includes specific additional materials utilized for the temperature, moisture, air flow, and biochemical properties inside the nose, the specific additional materials including at least one of purified water with liquid oxygen, sodium bicarbonate, sodium lauryl sulfate, poloxamer, alkylpolyglycosides, citric acid, pullulanase, and amylase.

14. The device of claim 13, wherein each tab is contiguous and able to be inserted into either or both nostrils without a connector and, when in use, the tab is not visible.

15. The device of claim 14, wherein the tab dissolves within 30 minutes or longer if predetermined otherwise for optimal dose and dissolve time.

16. The device of claim 15, wherein:
the scent, fragrance or medicine is pre-infused into the dissolvable tab;
a plurality of tabs are individually wrapped; and the plurality of wrapped tabs is inserted into a kit.
